(19) United States
(12) Patent Application Publication
(10) Pub. No.: US 2002/0103126 A1
(43) Pub. Date: Aug. 1, 2002

(54) Stable pharmaceutical form of administration for peptides, proteins and nucleic acids

(75) Inventors: Wolfgang Rodel, Heidelberg (DE); Markus Mattern, Heppenheim (DE); Gerhard Winter, Dossenheim (DE)

Correspondence Address: ARENT FOX KINTNER PLOTKIN & KAHN, 1050 CONNECTICUT AVENUE, N.W., SUITE 600, WASHINGTON, DC, 20036, US

(73) Assignee: Roche Diagnostics GmbH

(21) Appl. No.: 10/046,311

(22) Filed: Jan. 16, 2002

Related U.S. Application Data
(63) Continuation of application No. 09/741,196, filed on Dec. 21, 2000, now abandoned.
Application No. 09/741,196 is a continuation of application No. 09/403,021, filed on Jan. 6, 2000, now abandoned.
Application No. 09/403,021 is a 371 of international application No. PCT/EP98/02131, filed on Apr. 11, 1998.

(30) Foreign Application Priority Data
Apr. 18, 1997 (DE) 197 16 154.5

(51) Int. Cl. A61K 48/00; A61K 38/00; A61K 31/715

(52) U.S. Cl. 514/12; 514/44; 514/54

(57) ABSTRACT

The invention concerns storage-stable lyophilized pharmaceutical preparations of biomolecules wherein the biomolecules are selected from the group comprising proteins, peptides, nucleic acids and carbohydrates and additionally contain one or several basic D or L-amino acids in addition to one or several aminodicarboxylic acids, hydroxycarboxylic acids or dicarboxylic acids or physiologically compatible salts thereof. The auxiliary substances are present in the lyophilisate in a completely or partially amorphous form.

The invention concerns stable lyophilized compositions for pharmaceutical or diagnostic use which contain a protein, a peptide, a nucleic acid or a polysaccharide wherein the auxiliary substances are selected such-that the lyophilisates are present in an amorphous or partially amorphous form.

Advances in biotechnology in the past 20 years have resulted in an enormous increase in the number of biomolecules that are available in large amounts. A particularly active field for these products is their use in pharmaceutical therapies. Thus for example certain proteins are used to regulate individual cell types, nucleic acids are used to regulate gene expression and polysaccharides are used for vaccination. It is of practical advantage when the preparations can be stored at room temperature since refrigerated storage space is often limited.

Furthermore temperature-sensitive preparations require an accurate monitoring of the storage period between removal from the refrigerator and the therapeutic administration (e.g. injection) since byproducts can be formed as a result of degradation reactions which can adversely change their spectrum of action. It is difficult to ensure a continuous supervision of the storage conditions of the preparations especially in the hospital routine and when administering pharmaceutical preparations for example for clinical studies on humans.

All biomolecules can readily hydrolyse to a greater or lesser extent. Hydrolysis is part of the natural metabolism and is for example necessary to prevent the accumulation of high molecular toxic substances in the body.

Furthermore numerous other degradation reactions are described in the literature which affect biomolecules to varying degrees. In the case of peptides or proteins such degradation reactions occur by means of aggregation, denaturation, isomerization or redox processes. In the case of nucleic acids deamination or addition of a nucleophile for example results in the degradation of nucleic acids.

For the development of stable lyophilisates e.g. of pharmaceutical or diagnostic preparations of peptides or proteins, there are still no established causal methods which would allow those auxiliary substances to be reliably selected from a plurality of possible auxiliary substances and additives that would ensure a stable form of administration for the respective active substance. The selection of suitable auxiliary substances to make an adequately stable form of administration which for example ensure an adequately long storage stability or which delay or prevent the aforementioned degradation reactions is usually carried out empirically.

It is known that the storage stability of many protein preparations is increased by removing water. Suitable methods for this are freeze-drying and vacuum-drying. However, the use of such technical processes can also cause degradation reactions e.g. a freezing phase is necessary in freeze-drying. However, many proteins are not sufficiently resistant to freezing processes. When an aqueous solution of a biopolymer cools, most of the water crystallizes whereas the biopolymer remains in an amorphous state. This can lead to a change in the molecular environment of the biopolymer which can also result in a change in the spatial structure or conformation of the biopolymer. This can in turn favour degradation reactions by for example increasing the reactivity of individual functional groups or by aggregation of unfolded chain segments of adjacent polymer molecules. In addition removal of the hydrate envelope surrounding the protein in the drying phase can lead to chemical reactions such as oxidation in the protein chain. The addition of suitable additives can prevent or reduce the extent of these degradation reactions.

In the case of freeze-drying or vacuum-drying an important function of the auxiliary substances is to provide a stabilizing amorphous environment for the biopolymer which solidifies into a glass state on further cooling. The transition occurs in a step-like manner within a very narrow temperature interval and is characterized by the glass temperature Tg′. The molecular mobility and thus also the reactivity s greatly decreased below this temperature. In a formulation which is very suitable for freeze-drying, the Tg′ is as high as possible and typically above −40° C. The presence of amorphous structures can for example be demonstrated by differential scanning calorimetry (DSC), by X-ray diffraction examinations or by optical and electron-microscopic examinations.

In order to produce adequately stable lyophilized pharmaceutical forms of administration, it is necessary to select auxiliary substances which do not crystallize or at most only partially crystallize during the freezing. Such auxiliary substances which protect the biomolecule during the freezing process are named ‘cryoprotectants’. In the main drying phase the ice crystal sublime whereas in the afterdrying phase part of the water bound in the amorphous phase and in the biopoplymer is removed which usually requires more drastic conditions (higher temperature or a stronger vacuum). The Tg′ increases as the water content in the freeze-drying material decreases. In order to shorten the time for the drying process, the temperature of the plates of the freeze-drying chamber is successively increased but the temperature in the freeze-drying material must never exceed Tg′.

During the drying phase the auxiliary substances maintain the glass state in which the polymer is embedded. In addition the removal of water molecules in the afterdrying phase results in the formation of free valencies for hydrogen bonds in the biopolymer. This increases the reactivity of the biopolymer. The addition of suitable stabilizing auxiliary substances is intended to result in the formation of hydrogen bridges in order to create a water substitute environment for the biopolymer. The term ‘lyoprotectants’ was introduced for this.

The upper limit for temperature stress during storage is determined by the glass transition temperature Tg above which there is a pronounced increase in the molecular mobility. This often results in crystallization processes (described by the crystallization temperature Tk) or chemical reactions. Viewed macroscopically this often results in the so-called collapse of the lyophilisate cake (described by the collapse temperature Tc) since the molecules of the auxiliary substance associate with one another which is accompanied by a reduction of the specific surface of the auxiliary substance matrix.

The water present in the lyophilisate decreases Tg; in a good formulation the residual moisture content after freeze-drying is below 3%. However, it can increase somewhat during longer storage. In order to have an adequate safety margin, the storage temperature of the lyophilisate in the glass state should be at most 20° C. below Tg.

WO 93/00807 describes a two-component system composed of a cryoprotectant (such as polyethylene glycol, PVP or starch) or a lyoprotectant (such as sugar, polyhydroxy alcohol or amino acid) for stabilization during the lyophilization.

It is well-known that the tendency for class formation increases with the molecular weight. Hence polymers such as PVP, proteins (in particular serum albumin) or polysaccharides (Dextran) are used to form stable glass matrices.

However, it is known that protective proteins like serum albumin can be disadvantageous after injection since they can induce the formation of antibodies which impairs their use for parenteral preparations. In addition differences in the raw material batches of protective proteins can result in uncertainties since this can adversely influence the ability to process and the quality of the resulting product batches.

Moreover, polysaccharides used as auxiliary substances can have a pyrogenic effect in the blood circulation. An additional disadvantage of polysaccharides is that they often require swelling so that they often impede the rapid formation of a clear solution when a lyophilisate is reconstituted. In addition the material is usually composed of a fraction comprising different chain lengths which makes it more difficult to achieve a batch consistency. The latter also applies to synthetic polymers such as PVP (polyvinylpyrrolidone).

Previously polyhydroxy compounds such as saccharides (sucrose, trehalose, glucose) or sugar alcohols (mannitol) have been used almost exclusively as low molecular substances for glass formation in lyophilisates for biomolecules. However, the addition of mannitol only results in metastable glass states which can after-crystallize during storage.

Reducing sugars such as glucose or maltose can cause radical or redox reactions and also form Amadori products with primary amino groups (e.g. in proteins). In addition the Maillard reaction can result in a brown discolouration of the preparation. Non-reducing disaccharides or trisaccharides can hydrolyse which, on the one hand, can lead to the formation of reducing sugars, but on the other hand can potentially impair the physical properties of the auxiliary substance matrix. It is known that sugar alcohols like mannitol can catalyse hydrolysis reactions for example in the presence of acetate. In addition they have a tendency to crystallize. Nevertheless mannitol/glycine/(optionally phosphate, detergent) combinations are frequently used as an auxiliary substance matrix to lyophilize proteins (cf. EP 0 597 101, WO 89/09614).

Other disadvantages or problems in producing storage-stable sugar preparations that are really sufficiently dry are considerably increased drying periods since only a small heat input is possible due to the stability of the biological materials that are used. Long process times are economically unfavourable and in addition there is an increased process risk e.g. leaks can occur in the vacuum chamber, the cooling system can break down etc.

It was surprisingly found that certain combinations of auxiliary substances are suitable as glass formers for the lyophilization of biomolecules. Hence the present invention concerns lyophilized preparations containing a) biomolecules selected from the group comprising proteins, peptides, nucleic acids and carbohydrates and b) one or several basic D-amino acids or L-amino acids and c) one or several aminodicarboxylic acids, hydroxy-carboxylic acids, hydroxydicarboxylic acids or dicarboxylic acids or physiologically tolerated salts thereof wherein the auxiliary substances are present in the lyophilisate at least partially in an amorphous form. One or several neutral amino acids may also be optionally added to facilitate the drying and to improve the morphological structure of the lyophilisate cake.

The selection of the auxiliary substances has the effect that the auxiliary substances are present in the lyophilisates either completely amorphous or at least in a partially amorphous modification. In contrast to crystalline compositions, such lyophilisates have a glass transition temperature (Tg) which is above the intended storage temperature. Suitable combinations of auxiliary substances are mixtures containing at least one substance from each of the groups (A) and (B), wherein (A) is a basic D-amino acid or L-amino acid and (B) is an aminodicarboxylic acid and in particular an acidic D-amino acid or L-amino acid, aminocarboxylic acid, monocarboxylic acid, dicarboxylic acid or hydroxydicarboxylic acid or physiologically tolerated salts thereof. Such mixtures are suitable as glass formers for the lyophilization of biomolecules and as a result have the advantage that the lyophilisates prepared in this manner are stable for a long period which depends on the sensitivity of the biomolecule used and is preferably at least one year, in particular one to two years at refrigerated temperature or room temperature. This allows a reduction or complete avoidance of the use of the aforementioned less suitable groups of substances so that the disadvantages in using the said groups of substances can be largely avoided when producing pharmaceutical forms of administration. An additional advantage of the lyophilisates produced according to the invention is a great reduction in the drying time especially when using a hydrophobic amino acid of less than 30 hours, preferably less than 24 hours and in particular less than 15 hours. This means that the lyophilisates can be produced by drying overnight instead of a drying process which often requires several days.

Pharmaceutically stable lyophilisates can be obtained when the pair comprising the basic amino acid and the counterion required to adjust the pH are selected such that a matrix is formed during the lyophilization which is at least partially amorphous and has a glass transition temperature of more than 50° C., preferably more than 65° C. and in particular more than 80° C. It is advantageous for the production process when the frozen solution has a glass transition temperature of more than −40°C.

In order to adjust the pH value it is possible to additionally use physiologically tolerated acids or bases and salts thereof. Suitable acids are inorganic or organic acids such as phosphoric acid, acetic acid etc. Free acids or bases are preferably used in order to achieve a salt concentration in the lyophilisate which is as low as possible. In the case of some peptides and proteins the formation of protein aggregates was observed in arginine phosphate when preparing the solution to be lyophilized if the phosphate content was more than 5 mM. A similar behaviour was found when using arginine citrate (c=10 mM). Surprisingly the formation of aggregates can be reduced or largely avoided when monocarboxylic acids or dicarboxylic acids are used as a counterion instead of the phosphate salt of the amino acid arginine. Lyophilisates with a stable glass state were obtained in particular when aminodicarboxylic acids (e.g. acidic D-amino acids or L-amino acids) or dicarboxylic acids are used. Optionally phosphoric acid at a concentration of less than 5 mM can be used for the fine adjustment of the pH in the range of 5-7 when using a basic amino acid.

Moreover the forms of administration according to the invention have the additional advantage that the glass transition temperature as well as the appearance of the lyophilisate cake was further improved especially when a neutral amino acid was additionally added even if it partially crystallizes. The amount of this amino acid can be varied within wide limits (5-50% of the total amount of auxiliary substance).

The forms of administration according to the invention have the advantage that they are stable when stored for long periods at room temperature. Hence a safe use as a pharmaceutical preparation is guaranteed even if the cold chain is broken.

Suitable additives or auxiliary substances in the sense of the present invention are a combination of a basic, an acidic and at least one neutral amino acid in a preferred embodiment. These combinations are physiologically well-tolerated, have good freeze-drying properties and improve the thermal stability of lyophilized biopolymers. In addition the dissolution of the lyophilisate with water rapidly leads to a clear solution.

Basic amino acids that are suitable in the sense of the present invention are all physiologically tolerated amino acids with a basic side group such as histidine, lysine, arginine, ornithine or citrulline. Correspondingly suitable neutral amino acids are physiologically tolerated amino acids with hydrophobic or hydrophilic side groups such as phenylalanine, glycine, leucine or isoleucine. Suitable acids are corresponding aminodicarboxylic acids, hydroxycarboxylic acids, hydroxydicarboxylic acids, dicarboxylic acids or physiologically tolerated salts thereof such as aspartic or glutamic acid. If these acids have a chiral centre it is possible to use racemates or even optically active derivatives.

The amount of additives according to the invention is preferably selected such that the weight ratio of the acids mentioned in group c) (aminodicarboxylic acids, hydroxycarboxylic acids or dicarboxylic acids) to the basic D-amino acids or L-amino acids of group a) is in the range of 0.01:1 to 2:1 in the lyophilisate. A range of 0.1:1 to 1:1 and in particular about 0.5:1 is particularly advantageous.

Numerous peptides or proteins come into consideration within the sense of the present invention as active substances for the production of the pharmaceutical forms of administration according to the invention such as immunomodulators, lymphokines, monokines, cytokines, enzymes, antibodies, growth factors, growth-inhibiting factors, blood proteins, hormones, vaccines, blood coagulation factors and corresponding precursor proteins, muteins or fragments thereof. The peptides or proteins have a molecular weight of 0.5-500 kD, preferably 2.0-200 kD. The following peptides or proteins are mentioned as examples: atrial naturetic factor or ANP (cf. WO 85/33768), urodilatin or ularitide (cf. WO 88/06596, WO 95/33768), cardiodilatin (cf. WO 85/02850), BNP (brain natriuretic peptides), auriculin, interferons, colony-stimulating factors, interleukins (IL-1, IL-1α, IL-1β,IL-2, IL-3, IL-4, etc.) macrophage-activating factors, B-cell factors, urokinase, plasminogen activators, TNF, NGF; erythropoietin, EGF, hGH, BMP (bone morphogenic proteins), calcitonin, insulin or relaxin.

Nucleic acids such as plasmids, DNA fragments or RNA strands are also suitable for the inventive form of administration.

The lyophilized pharmaceutical forms of administration according to the invention are particularly suitable for parenteral administration in a liquid. form.

The invention is illustrated by the following examples and comparative examples and is elucidated in the following. Formulations have been found which, as glass formers in the lyophilization of biomolecules, considerably increase the glass transition temperature, largely prevent aggregation of the biomolecules, improve the appearance of the lyophilisate cake and are suitable for the thermal stabilization of lyophilized biomolecules. The listed formulations demonstrate that only the mixtures according to the invention lead to the desired result, i.e. they enable stable protein formulations to be obtained in a short lyophilization process as completely or partially amorphous structures.

The concentrations stated in the formulations of the following examples relate to the solution before lyophilisation.

### EXAMPLE 1

| | Concentration of |
|---|---|
| | the initial |
| Formulation 1 | solution |
| L-arginine | 20 mg/ml |
| aspartic acid | 10 mg/ml |
| Tween 80 | 0.1 mg/ml |
| G-CSF | 0.35 mg/ml |

The pH value of the solution is adjusted to pH 7.4 with H₃PO₄.

2 g L-arginine and 1 g L-aspartate were dissolved in 50 ml water. 35 mg G-CSF (dissolved in 30 ml 10 mM phosphate buffer) was added to this solution by pipette and stirred for 5 min. Subsequently 100 μl Tween 80 (as a 10% aqueous solution) was added by pipette and it was stirred for a further 20 minutes. The pH was adjusted to 7.4 by addition of phosphoric acid and the volume was made up to 100 ml. This solution was filtered through a membrane (PVDF filter 0.22 μm) and 1 ml aliquots were dispensed into glass vials. They were freeze-dried after putting on a suitable stopper and the drying was carried out for a total period of 40 hours. The vials were subsequently sealed and stored at different temperatures until analysis. It was found by DSC that the glass transition temperature of the cake was 95° C. After 26 weeks X-ray diffraction spectra were recorded from samples of this lyophilisate that were stored under different conditions. These spectra show that it is amorphous even after storage at a temperature of +60° C.

### EXAMPLE 2

### Comparative Example

| | Concentration of |
|---|---|
| | the initial |
| Formulation 2 | solution |
| L-valine | 20 mg/ml |
| glycine | 20 mg/ml |
| Tween 80 | 0.1 mg/ml |
| G-CSF | 0.35 mg/ml |

The pH value of the solution is adjusted to pH 7.4 with NaOH.

2 g L-valine and 2 g glycine were dissolved in 50 ml water. 100 μl Tween 80 (as a 10% aqueous solution) was added by pipette and it was stirred for a further 20 minutes. The pH was subsequently adjusted to 7.4 by addition of NaOH. 35 mg G-CSF (dissolved in 30 ml 10 mM phosphate buffer) was added by pipette to this solution and stirred for 5 min. The pH was checked and the volume was made up to 100 ml. After filling into vials a lyophilisate was prepared from this solution as in example 1. No glass transition can be seen in the DSC of this lyophilisate immediately after its preparation. X-ray diffraction spectra and images in the scanning electron microscope showed that the cake is completely crystalline. No amorphous or partially amorphous structures are detectable.

### EXAMPLE 3

### Comparative Example

| | Concentration of |
|---|---|
| | the initial |
| Formulation 3 | solution |
| L-valine | 10 mg/ml |
| glycine | 20 mg/ml |
| Tween 80 | 0.1 mg/ml |
| LDH | (150 U^{25° C.}) 0.3 mg |

The pH value of the solution is adjusted to pH 7.4 with NaOH. 1 g L-valine and 2 g glycine were dissolved in 70 ml water, 100 μl Tween 80 (as a 10 % aqueous solution) was added and it was stirred for 20 minutes. Subsequently the pH was adjusted to 7.0 by addition of NaOH. 30 mg (15 kU) LDH (from porcine muscle, dissolved in 20 ml 20 mM phosphate buffer) was added by pipette to this solution and stirred for 5 min. The pH was checked and the volume was made up to 100 ml. A lyophilisate was prepared from this solution after filling into vials as described in example 1. This formulation is also completely crystalline. No amorphous structures are detectable.

### EXAMPLE 4

| Formulation 4 | Amount per vial |
|---|---|
| L-arginine | 20 mg |
| L-phenylalanine | 10 mg |
| L-aspartic acid | 10 mg |
| Tween 80 | 0.1 mg |
| LDH | (150 U^{25° C.}) 0.3 mg |
| (from porcine muscle) | |

The pH value of the solution is adjusted to pH 7.4 with H₃PO4.

2 g L-arginine, 1 g aspartic acid and 1 g L-phenylalanine were dissolved in 70 ml water, 100 μl Tween 80 (as a 10% aqueous solution) was added and it was stirred for 20 minutes. Subsequently the pH was adjusted to 7.4 by addition of phosphoric acid. 50 mg (15 kU) LDH (from porcine muscle, dissolved in 30 ml 100 mM phosphate buffer) was added by pipette to this solution and stirred for 5 min. The pH was checked and the volume was made up to 100 ml. A lyophilisate was prepared from this solution after filling into vials as described in example 1. The analysis showed that some of the phenylalanine was present in a crystalline form i.e. the cake is partially crystalline and partially amorphous. The crystalline proportion remained constant during storage.

### EXAMPLE 5

The content of unchanged G-CSF in formulations 1 and 2 was determined by RP-HPLC after storing the vials at room temperature (RT):

| | Proportion of |
|---|---|
| | unchanged protein |
| | after 4 weeks RT |
| bulk active substance without | 96.5% |
| addition of auxiliary | |
| substances, lyophilized | |
| L-arginine/phosphate/aspartic | 98.9% |
| acid/Tween 80 (formulation 1) | |
| L-valine/glycine/Tween 80 | 92.0% |
| (formulation 2) | |

The enzymatic activity of formulations 3 and 4 was determined in a coupled optical test after storing the vials at RT (after 5 or 13 weeks):

| | after lyo- | 5 weeks | 13 weeks |
|---|---|---|---|
| | philization | RT | RT |
| bulk active substance | 61.3% | 29.1% | 11.4% |
| without addition of | | | |
| auxiliary substances, | | | |
| lyophilized | | | |
| L-arginine/phosphate/ | 81.2% | 74.3% | 58.1% |
| aspartic acid/L-phenyl- | | | |
| alanine (formulation 4) | | | |
| L-valine/glycine/Tween 80 | 64.1% | 9.2% | <1% |
| (formulation 3) | | | |

### EXAMPLE 6

Lyophilisates were prepared in an analogous manner to formulation 4, but arginine was replaced by the same molar amount of other basic aminocarboxylic acids. The enzymatic activity of LDH in the lyophilisate was determined after 5 weeks storage at room temperature (RT).

| | | after | |
|---|---|---|---|
| | No. | lyophilization | 5 weeks RT |
| L-citrulline | form. 5 | 82.2% | 60.4% |
| L-histidine | form. 6 | 65.5% | 53.1% |
| L-lysine | form. 7 | 68.6% | 56.5% |
| L-ornithine | form. 8 | 65.5% | 46.5% |

### EXAMPLE 7

| | Concentration of the |
|---|---|
| Formulation | initial solution |
| L-arginine | 20 mg/ml |
| L-isoleucine | 10 mg/ml |
| rhNGF | 10 μg |

The pH value of the solution is adjusted to pH 6.3 with acid (see below).

2 g L-arginine was dissolved in 50 ml water and a pH of 6.3 was adjusted by adding an acid. 1 g isoleucine and 1 mg rhNGF were added and made up to a volume of 100 ml with water. This solution was filtered through a membrane (PVDF filter 0.22 μm) and 1 ml aliquots were dispensed into glass vials. After putting on a suitable stopper, a freeze-drying was carried out after a total drying period of about 40 hours. The lyophilisates were subsequently measured using DSC.

Result:

| Acid used to adjust | | | |
|---|---|---|---|
| the pH | No. | Tg | Evaluation |
| HCl | form. 9 | 41.4° C. | − |
| L-lactic acid | form. 10 | 54.3° C. | (+) |
| 3-hydroxybutyric acid | form. 11 | 37.7° C. | − − |
| malic acid | form. 12 | 67.8° C. | + |
| succinic acid | form. 13 | 76.1° C. | + |
| fumaric acid | form. 14 | 81.0° C. | + + |
| maleic acid | form. 15 | 82.3° C. | + + |
| L-glutamic acid | form. 16 | 86.1° C. | + + |

### EXAMPLE 8

### Comparative Example

| | Concentration of the |
|---|---|
| Formulation 17 | initial solution |
| L-arginine | 20 mg/ml |
| ularitide | 1.0 mg/ml |

The pH value of the solution is adjusted to pH 6.0 with H₃PO₄.

2 g L-arginine was dissolved in 50 ml water and a pH of 6.0 was adjusted by addition of phosphoric acid. 100 mg ularitide (dissolved in 30 ml H₂O) was added by pipette to this solution and it was stirred for 10 min. The solution became turbid after 60 minutes and the protein flocculated. After that the experiment was terminated.

### EXAMPLE 9

### Comparative Example

| | Concentration of the |
|---|---|
| Formulation 18 | initial solution |
| L-arginine | 20 mg/ml |
| ularitide | 1.0 mg/ml |

The pH value of the solution is adjusted to pH 6.0 with citric acid.

2 g L-arginine was dissolved in 50 ml water and a pH of 6.0 was adjusted by addition of citric acid. 100 mg ularitide (dissolved in 30 ml H₂O) was added by pipette to this solution and it was stirred for 10 min. The solution became turbid after 2 hours and the protein flocculated. After that the experiment was terminated.

### EXAMPLE 10

Lyophilisates containing the active substance ularitide were prepared by the process from example 7 with the following compositions per vial:

a) Formulation 19 (comparative example)

1 mg ularitide

10 mg mannitol

adjust to pH 6.3 with acetic acid

Analysis of the X-ray diffraction pattern showed that the lyophilisate has a completely crystalline structure. No amorphous or partially amorphous structures are detectable.

b) Formulation 20

1 mg ularitide

1-20 mg L-arginine

10 mg L-isoleucine

adjust to pH 6.3 with aspartic acid

Evaluation in DSC:

formulation 19: no glass transition is detectable, completely crystalline

formulation 20: Tg=85.1° C., partially amorphous structure

Both formulations 19 and 20 were stored for 1 year at room temperature. The results of the subsequent gel electrophoresis were as follows: form. 19: dimers>1% and soluble aggregates form. 20: 100% monomer.

### EXAMPLE 11

Lyophilisates with the following formulation compositions per vial were prepared using the process from example 7:

a) Form. 21 (comparative example)

1 mg ularitide

50 mg sucrose

10 mg glycine

6 mg polyethylene glycol 6000

b) Form. 22

1 mg-ularitide

20 mg L-arginine

10 ml L-isoleucine

adjust to pH 6.3 with aspartic acid

b) Form. 23

4 mg ularitide

20 mg L-arginine

10 ml L-isoleucine

adjust to pH 6.3 with aspartic acid

b) Form. 24

1 mg ularitide

20 mg L-arginine

10 ml L-isoleucine

adjust to pH 6.3 with aspartic acid

b) Form. 25 (comparative example)

1 mg ularitide

25 mg sucrose

20 mg glycine

All formulations 21-25 were stored in a stress test at different temperatures and subsequently the content was determined with RP-HPLC.

| | initial | 13 weeks | 13 weeks | 13 weeks |
|---|---|---|---|---|
| | value | 5° C. | 25° C. | 40° C. |
| Form. 21 | 99.3% | 99.1% | 98.8% | 96.3% |
| Form. 22 | 99.7% | 99.3% | 99.7% | 99.6% |
| Form. 23 | 99.6% | 99.6% | 99.5% | 99.2% |
| Form. 24 | 99.7% | 99.5% | 99.2% | 99.4% |
| Form. 25 | 99.4% | 99.5% | 98.2% | 93.1% |

The results of examples 10 and 11 show that the formulations according to the invention stabilize the peptide better against temperature stress than the corresponding formulations based on sucrose or sucrose/PEG of the prior art. The mannitol used in many formulations also does not result in an adequately stable formulation.

Examples 8 and 9 show that the acids that are often used in buffer systems lead to flocculation of certain peptides. This was not observed with the acids (preferably aspartic acid and glutamic acid) used in the formulations according to the invention.

### EXAMPLE 12

Lyophilisates containing the active substance ularitide were prepared by the process from example 7 with the following compositions per vial:

a) Form. 26 (comparative example)

1 mg ularitide

15 mg glycine

2 mg L-isoleucine

10 mg urea

0.5 mg polysorbate

adjust to pH 6.8 with Na acetate buffer; completely crystalline.

b) Form. 27

1 mg ularitide

20 mg D-arginine

10 mg D-isoleucine

adjust to pH 6.8 with D-aspartic acid

c) Form. 28

1 mg ularitide

20 mg L-arginine

10 mg L-isoleucine

adjust to pH 6.8 with L-aspartic acid

d) Form. 29 (comparative example)

1 mg ularitide

20 mg L-threonine

10 mg D-isoleucine

e) Form. 30 (comparative example)

1 mg ularitide

15 mg polyethylene glycol 6000

5 mg phenylalanine

The formulations were stored in a stress test at different temperatures and subsequently the amount of the main degradation product was determined by RP-HPLC (peak X1):

| | Initial | | | |
|---|---|---|---|---|
| | value | 13 weeks 5° C. | 13 weeks 40° C. | Tg |
| form. 26 | 0.2% | 2.1% | 6.5% | — |
| form. 27 | 0.4% | 0.4% | 0.5% | 83.8° C. |
| form. 28 | 0.2% | 0.2% | 0.4% | 84.6° C. |
| form. 29 | 0.3% | 1.8% | 3.5% | 61.2° C. |
| form. 30 | 0.3% | 2.8% | 4.7% | — |

Considerably less degradation product of the peptide is formed in the formulations according to the invention. Although formulation 29 results in an amorphous lyophilisate cake, it is not sufficient to stabilize the protein.

### EXAMPLE 13

Lyophilisates containing the active substance ularitide were prepared using the process from example 7 with the following compositions per vial:

a) Form. 31

1 mg ularitide

70 mg sucrose

10 mg L-phenylalanine

b) Form. 32

1 mg ularitide

85 mg sucrose

c) Form. 33

1 mg ularitide

46 mg raffinose

10 mg L-phenylalanine

d) Form. 34

1 mg ularitide

20 mg L-arginine

5 mg L-phenylalanine

adjust to pH 6.3 with L-aspartic acid

All formulations 31-34 were stored in a stress test at different temperatures, subsequently the lyophilisates were each dissolved in 1 ml water and the turbidity of the reconstituted solution was determined after 1 hour in a nephelometer (type Hach). Turbidity values above 1.0 are rated as unacceptable. Since the auxiliary subtances are readily soluble, the turbidity observed in several formulations is due to an aggregation of the pepide.

Results:

| Form. | Initial | 4 w. | 4 w. | 4 w. | 13 w. | 13 w. | 13 w. |
|---|---|---|---|---|---|---|---|
| No. | value | KS | RT | 40° C. | KS | RT | 40° C. |
| 21 | 0.6 | 0.8 | 0.5 | 1.6 | 0.8 | 1.4 | 3.6 |
| 25 | 0.7 | 0.5 | 0.4 | 1.2 | 0.8 | 0.9 | 1.4 |
| 31 | 0.7 | 0.7 | 0.6 | 0.6 | 1.4 | 1.7 | 1.7 |
| 32 | 0.8 | 0.7 | 1.1 | 1.0 | 1.7 | 1.9 | 1.7 |
| 33 | 1.4 | 1.2 | 1.1 | 1.4 | n.d. | n.d. | n.d. |
| 34 | 0.6 | 0.5 | 0.7 | 0.6 | 0.5 | 0.5 | 0.5 |
| 22 | 0.5 | 0.6 | 0.6 | 0.7 | 0.6 | 0.7 | 0.8 |

KS = refrigerator temperature: −8° C.
RT = room temperature: 20-22° C.

Only the formulations according to the invention did not exceed the turbidity threshold value of 1.0 after thermal stress.

### EXAMPLE 14

Ularitide was reconstituted in various formulations after storage as in example 11 and the solution was examined for particles using a light-scattering measuring instrument (PMS).

Results: (in each case the means from counting 5 vials are stated)

| | Initial | | 4 weeks | | 4 weeks | | 13 weeks | | 13 weeks | |
|---|---|---|---|---|---|---|---|---|---|---|
| Form | value | | KS | | 40° C. | | KS | | 40° C. | |
| No. | >5 μm | >25 μm | >5 μm | >25 μm | >5 μm | >25 μm | >5 μm | >25 μm | >5 μm | >25 μm |
| 26 | 78 | 4 | 140 | 21 | 1262 | 28 | 713 | 28 | 2560 | 35 |
| 33 | 36 | 2 | 160 | 6 | 849 | 17 | 440 | 11 | 1389 | 22 |
| 34 | 4 | 0 | 6 | 0 | 21 | 4 | 31 | 1 | 65 | 11 |
| 22 | 11 | 0 | 13 | 1 | 34 | 3 | 17 | 0 | 42 | 7 |

The formulations according to the invention show no critical increase of the number of particles after storage at an elevated temperature.

### EXAMPLE 15

Lyophilisates containing the protein rhNGF were prepared with the following compositions:

a) Form. 35

0.025 mg rhNGF

20 mg L-arginine

10 mg L-aspartic acid

adjust to pH 6.3 with acetic acid

b) Form. 36

0.025 mg rhNGF

20 mg L-arginine

10 mg L-aspartic acid

10 mg L-isoleucine

adjust to pH 6.3 with acetic acid

c) Form. 37 (comparative example)

0.025 mg rhNGF

20 mg L-arginine

30 mg sucrose

adjust to pH 6.3 with acetic acid

d) Form. 38 (comparative example)

0.025 mg rhNGF

20 mg L-arginine

30 mg raffinose

adjust to pH 6.3 with acetic acid

The lyophilisation programme was considerably shortened compared to the usual programmes: a total of 15 hours instead of usually 40-50 hours. Subsequently the residual moisture in the lyophilisate was determined in two independent determinations.

Result:

| | |
|---|---|
| Formulation 35 | 4.7%/5.0% |
| Formulation 36 | 1.0%/0.9% |
| Formulation 37 | 5.9%/6.6% |
| Formulation 38 | 5.6%/5.6% |

The example shows that a shortening of the lyophilisation period is only possible with the formulations according to the invention especially when using-a hydrophobic amino acid as the third component.

### EXAMPLE 16

Lyophilisates containing the protein rhNGF were prepared with the following formulations:

a) Form. 39

0.025 mg rhNGF

20 mg L-arginine

10 mg β-alanine

10 mg L-aspartic acid

adjust to pH 6.3 with acetic acid

b) Form. 40

0.025 mg rhNGF

20 mg L-arginine

8 mg L-aspartic acid

10 mg L-isoleucine

adjust to pH 8.7 with acetic acid

c) Form. 41

0.025 mg rhNGF

20 mg L-arginine

12 mg L-aspartic acid

10 mg L-isoleucine

adjust to pH 4.5 with malic acid

The lyophilisation was carried out with the same programme as stated in example 15 and subsequently the residual moisture was determined:

| Result: | |
|---|---|
| Formulation 39 | 1.2%/1.4% |
| Formulation 40 | 1.6%/1.9% |
| Formulation 41 | 0.8%/0.9% |

1. Lyophilized preparations containing
a) biomolecules selected from the group comprising proteins, peptides, nucleic acids and carbohydrates
b) one or several basic D-amino acids or L-amino acids and
C) one or several aminodicarboxylic acids, hydroxy-carboxylic acids, hydroxydicarboxylic acids or dicarboxylic acids
or physiologically compatible salts thereof wherein the auxiliary substances are present in the lyophilisate in a completely or partially amorphous form.

2. Lyophilized preparations as claimed in claim 1, wherein the weight ratio of the additives mentioned in c) to the additives mentioned in b) is in the range of 0.01:1 to 2:1.

3. Lyophilized preparations as claimed in one of the claims 1-2, wherein they contain polymers (compounds with a molecular weight>1000 Da) as auxiliary substances in an amount of less than 10% by weight of the total mass of auxiliary substances.

4. Lyophilized preparations as claimed in one of the claims 1-3, wherein they contain sugars as auxiliary substances in an amount of less than 10% by weight of the total mass of auxiliary substances.

5. Lyophilized preparations as claimed in one of the claims 1-4, wherein they have a glass transition temperature of>50° C.

6. Lyophilized preparations as claimed in one of the claims 1-5, wherein the frozen solution has a glass transition temperature above −40° C. before lyophilization.

7. Lyophilized preparation as claimed in one of the claims 1-6, wherein the aminocarboxylic acid is aspartic acid or glutamic acid.

8. Lyophilized preparations as claimed in one of the claims 1-7, wherein they additionally contain one or several amino acids with a hydrophobic residue.

9. Lyophilized preparations as claimed in claim 8 containing leucine, isoleucine, valine or phenylalanine, in particular isoleucine or phenylalanine.

10. Lyophilized preparations as claimed in one of the claims 1-9, wherein the solution reconstituted with water has a pH between about 3 and 9.

11. Lyophilized preparations as claimed in one of the claims 1-10, wherein the mass ratio of biomolecule to auxiliary substance is less than 1:10.

12. Lyophilized preparation as claimed in one of the claims 1-11, wherein the biomolecule is a peptide from the class of atrial natriuretic peptides (ANP).

13. Process for the production of lyophilized preparations as claimed in one of the claims 1-12, wherein a solution or suspension of the biomolecule is prepared in a physiologically tolerated solvent and a) one or several basic D-amino acids or L-amino acids and b) at least one or several aminodicarboxylic acids or organic or inorganic acids are added and subsequently the solution is lyophilized.

14. Process as claimed in claim 13, wherein the lyophilization is carried out starting with an aqueous solution with a phosphate content of less than 5 mM.
